(19) United States
(12) Patent Application Publication
(10) Pub. No.: US 2012/0238725 A1
(43) Pub. Date: Sep. 20, 2012

(54) PRIMARY CARBON NANOPARTICLES

(75) Inventors: Robert Elghanian, Wilmette, IL (US); Zaheer Parpia, Evanston, IL (US)

(73) Assignee: Northwestern University, Evanston, IL (US)

(21) Appl. No.: 13/393,847

(22) PCT Filed: Sep. 3, 2010
(86) PCT No.: PCT/US10/47892

§ 371 Date: May 16, 2012

Related U.S. Application Data
(60) Provisional application No. 61/240,067, filed on Sep. 4, 2009.
(60) Provisional application No. 61/361,754, filed on Jul. 6, 2010.

(30) Foreign Application Priority Data
Sep. 3, 2010 (US) PCT/US2010/047892

(51) Int. Cl. B32B 5/16; C01B 31/02; C07K 17/14; B82Y 40/00; B82Y 30/00

(52) U.S. Cl. 530/345; 428/402; 423/445 R; 530/391.1; 530/402; 977/773; 977/896

(57) ABSTRACT

The present invention provides monodisperse primary carbon nanoparticles, and methods of preparation and use thereof. In particular, the present invention provides surface-modified monodisperse primary carbon nanoparticles, and methods of preparation and use thereof.

### CROSS-REFERENCE TO RELATED APPLICATION

The present invention claims priority to U.S. Provisional Patent Application Ser. No. 61/240,067 filed Sep. 4, 2009, and U.S. Provisional Patent Application Ser. No. 61/361,754 filed Jul. 6, 2010, which are hereby incorporated by reference in their entireties.

### FIELD OF THE INVENTION

The present invention provides monodisperse primary carbon nanoparticles, and methods of preparation and use thereof. In particular, the present invention provides surface-modified monodisperse primary carbon nanoparticles, and methods of preparation and use thereof.

### BACKGROUND

Lateral flow assays take advantage of concentrating the analyte of interest in a detection zone with immobilized capture reagents on nitrocellulose. The presence of the analyte is verified by its capture at the detection zone using particles of high extinction coefficient that are conjugated to monoclonal antibodies and form the antigen antibody sandwich by flowing through the pores of nitrocellulose at the capture zone. A number of technologies have been developed that use gold, fluorescent latex, quantum dots, colored latex as well as carbon particles.

Previous attempts at using carbon nanoparticles have been focused on the use of small primary particle sizes (25 nm-60 nm) that may form a range of larger aggregates during the manufacturing of carbon black. As one can imagine it would take ten or more 25 nm particles strung together to make a 250 nm particle. An aggregate containing two or more primary particles is susceptible to disaggregation depending on the environment that it may encounter. In addition, use of small particles to generate larger ones leads to very high polydispersity. The aggregation of small primary particles is a highly variable process depending on the nature of the solvent, presence of other solutes, extent of sonication during suspension as well as the length and temperature of storage. Oxididation of aggregate carbon particles results in varying amounts of surface oxidation as can be demonstrated by XPS analysis of commercially available materials.

Carbon black is virtually pure elemental carbon in the form of colloidal particles that are produced by incomplete combustion or thermal decomposition of gaseous or liquid hydrocarbons under controlled conditions. Its physical appearance is that of a black, finely divided pellet or powder. Its use in tires, rubber and plastic products, printing inks and coatings is related to properties of specific surface area, particle size and structure, conductivity and color. Carbon black is also in the top 50 industrial chemicals manufactured worldwide, based on annual tonnage. All carbon blacks have chemisorbed oxygen complexes (i.e., carboxylic, quinonic, lactonic, phenolic groups and others) on their surfaces to varying degrees depending on the conditions of manufacture. These surface oxygen groups are collectively referred to as volatile content. It is also known to be a non-conductive material due to its volatile content. Certain grades of carbon black are produced through acid oxidation. Acid is sprayed in high temperature dryers during the manufacturing process to change the inherent surface chemistry of the black. The amount of chemically-bonded oxygen on the surface area of the black is increased to enhance performance characteristics.

### SUMMARY OF THE INVENTION

In some embodiments, the present invention provides a composition comprising monodisperse primary carbon nanopartices (PCN). In some embodiments, the present invention provides a composition comprising monodisperse PCN with diameters above about 100 nm. In some embodiments, the PCN have a polydispersity index of less than 0.5. In some embodiments, the PCN have a polydispersity index of less than 0.4. In some embodiments, the PCN have a polydispersity index of less than 0.3. In some embodiments, the PCN have a polydispersity index of less than 0.2. In some embodiments, the PCN have a polydispersity index of about 0.25. In some embodiments, the PCN have a polydispersity index of about 0.2. In some embodiments, the PCN have a polydispersity index of about 0.15. In some embodiments, the PCN have a polydispersity index of about 0.1. In some embodiments, the PCN are 100 nm or greater in diameter. In some embodiments, the PCN are 200 nm or greater in diameter. In some embodiments, the PCN are 300 nm or greater in diameter. In some embodiments, the PCN are less than 500 nm in diameter. In some embodiments, the PCN are less than 600 nm in diameter. In some embodiments, the PCN are less than 700 nm in diameter. In some embodiments, the PCN are less than 1000 nm in diameter. In some embodiments, the PCN have an average diameter of about 350 nm. In some embodiments, the PCN have an average diameter of about 300-400 nm. In some embodiments, the PCN comprise an oxidized surface. In some embodiments, the PCN are surface functionalized. In some embodiments, the PCN are surface functionalized with one member of a binding pair. In some embodiments, the surfaces of the PCN are functionalized with one or more biomolecules. In some embodiments, the biomolecules comprise antibodies. In some embodiments, the biomolecules are attached to the PCN by a linker.

In some embodiments, the present invention provides a method of producing monodisperse PCN comprising: (a) providing: (i) a carbon reagent; (ii) an oxidizing agent; (iii) a strong acid; and (iv) a basic quenching agent; (b) mixing the carbon reagent, oxidizing agent, and strong acid; (c) heating the mixture; and (d) quenching the reaction with the basic quenching agent. In some embodiments, the carbon reagent comprises carbon black. In some embodiments, the carbon black comprises thermal black. In some embodiments, the oxidizing agent comprises hydrogen peroxide. In some embodiments, the strong acid comprises nitric acid. In some embodiments, the basic quenching agent comprises sodium bicarbonate. In some embodiments, heating the mixture comprises heating to about 85° C. In some embodiments, heating the mixture comprises heating to about 80-90° C. In some embodiments, heating the mixture comprises heating to above 40° C., 50° C., 60° C., 70° C., 80° C. or 90° C.

In some embodiments, the present invention provides monodisperse PCN produced by a method comprising: (a) providing: (i) a carbon reagent; (ii) an oxidizing agent; (iii) a strong acid; and (iv) a basic quenching agent; (b) mixing the carbon reagent, oxidizing agent, and strong acid; (c) heating the mixture; and (d) quenching the reaction with the basic quenching agent. In some embodiments, the carbon reagent comprises carbon black. In some embodiments, the carbon black comprises thermal black. In some embodiments, the oxidizing agent comprises hydrogen peroxide. In some embodiments, the strong acid comprises nitric acid. In some embodiments, the basic quenching agent comprises sodium bicarbonate. In some embodiments, heating the mixture comprises heating to about 85° C. In some embodiments, heating the mixture comprises heating to about 80-90° C. In some embodiments, heating the mixture comprises heating to above 40° C., 50° C., 60° C., 70° C., 80° C. or 90° C. In some embodiments, the PCN have a polydispersity index of less than 0.5. In some embodiments, the PCN have a polydispersity index of less than 0.4. In some embodiments, the PCN have a polydispersity index of less than 0.3. In some embodiments, the PCN have a polydispersity index of less than 0.2. In some embodiments, the PCN have a polydispersity index of about 0.25. In some embodiments, the PCN have a polydispersity index of about 0.2. In some embodiments, the PCN have a polydispersity index of about 0.15. In some embodiments, the PCN have a polydispersity index of about 0.1. In some embodiments, the PCN are 100 nm or greater in diameter. In some embodiments, the PCN are 200 nm or greater in diameter. In some embodiments, the PCN are 300 nm or greater in diameter. In some embodiments, the PCN are less than 500 nm in diameter. In some embodiments, the PCN are less than 600 nm in diameter. In some embodiments, the PCN are less than 700 nm in diameter. In some embodiments, the PCN are less than 1000 nm in diameter. In some embodiments, the PCN have an average diameter of about 350 nm. In some embodiments, the PCN have an average diameter of about 300-400 nm. In some embodiments, the PCN comprise an oxidized surface. In some embodiments, the PCN are surface functionalized. In some embodiments, the PCN are surface functionalized with one member of a binding pair. In some embodiments, the surfaces of the PCN are functionalized with one or more biomolecules. In some embodiments, the biomolecules comprise antibodies. In some embodiments, the biomolecules are attached to the PCN by a linker.

In some embodiments, the present invention provides a method of surface-functionalizing a monodisperse primary carbon nanoparticle comprising reacting surface-oxidized monodisperse primary carbon nanoparticles with one or more functional molecules at elevated temperature. In some embodiments, the surface-oxidized monodisperse primary carbon nanoparticles comprise surface-oxidized thermal black. In some embodiments, the elevated temperature is about 55° C. or greater. In some embodiments, the elevated temperature is about 55° C. In some embodiments, the elevated temperature is about 55-100° C., about 55-85° C., about 55-65° C., about 60° C., about 65° C., about 70° C., about 75° C., about 80° C., about 85° C., about 90° C., about 95° C., etc. In some embodiments, the elevated temperature is a temperature above physiological temperature. In some embodiments, the functional molecule comprises a biomolecule. In some embodiments, the elevated temperature is a temperature above the physiological temperature of the species or the hybridoma within which the biomolecule (e.g. antibody) to be conjugated to the primary carbon nanoparticle was derived. In some embodiments, the biomolecule comprises a protein, peptide, or polypeptide. In some embodiments, the biomolecule is directly conjugated to the surface-oxidized monodisperse primary carbon nanoparticles. In some embodiments, the biomolecule is conjugated to the surface-oxidized monodisperse primary carbon nanoparticles by a linker. In some embodiments, the biomolecule comprises an antibody. In some embodiments, the present invention provides a surface-functionalized primary carbon nanoparticle prepared by the method described herein.

### BRIEF DESCRIPTION OF THE FIGURES

The following drawings form part of the present specification and are included to further illustrate aspects of the present invention. The drawings highlight exemplary embodiments of the present invention, but should not be viewed as limiting the scope of the invention. The invention may be better understood by reference to the drawings in combination with the detailed description of the specific embodiments presented herein.

FIG. 1 shows (a) a TEM image of Sp-15 particles prior to conjugation, and (b) a histogram of particle size distribution.

FIG. 2 shows a TEM of Sp15 particles post conjugation at 55° C.

FIG. 3 shows plots of dynamic light scattering data (a) oxidized carbon nanoparticles conjugates and (b) non-oxidized carbon nanoparticles conjugates after conjugation at 25° C. and 55° C.

FIG. 4 shows a comparison of performance of the SP15 particles prepared at 25° C. and 55° C. with and without oxidation.

FIG. 5 shows storage characteristics of oxidized particles prepared at 25° C. and 55° C. using identical buffers after standing at 4° C. Tube on the right shows almost complete precipitation of the conjugate prepared at 25° C. Tube on the left shows the black suspension under identical storage conditions.

FIG. 6 shows a plot of the effect of heating on non functional conjugates prepared at 25° C. and subsequently heated for two hours at 55° C.

FIG. 7 shows conjugation to MAb at 25° C. and 55° C. of (a) unoxidized and (b) oxidized carbon nanoparticles.

FIG. 8 shows EPR spectra of carbon nanoparticles.

FIG. 9 shows results of lateral flow assays performed on surface-conjugated particles prepared at a range of temperatures.

FIG. 10 shows the results of a PSA assay using smPCN.

FIG. 11 shows reduced background signal following conjugation by hot start method.

### DEFINITIONS

As used herein, the term “nanoparticle” refers to particles, groups of particles, particulate molecules (e.g. small individual groups of loosely associated groups of molecules), and groups of particulate molecules that while potentially varied in specific geometric shape have an effective, or average, diameter that can be measured on a nanoscale (e.g. measured in nanometers (nm) to hundreds of nanometers, generally less than 1 micron).

As used herein, the term “microparticle” refers to particles, groups of particles, particulate molecules (e.g. small individual groups of loosely associated groups of molecules), and groups of particulate molecules that while potentially varied in specific geometric shape have an effective, or average, diameter that can be measured on a microscale (e.g. measured in micrometers (μm) to hundreds of micrometers, generally less than 1 milimeter).

As used herein, the term “aggregate carbon nanoparticle” or “ACN” refers to a nanoparticle composed of multiple smaller particles (e.g. nanoscale or sub-nanoscale particles). An aggregate carbon nanoparticle is typically on the large end of the nanoscale and composed of several (e.g. 2-11), dozens (e.g. 12-99), or even hundreds (e.g. 100+) of smaller particles. The aggregated particles may be held together by physical and/or chemical forces.

As used herein, the term “primary carbon nanoparticle” or “PCN” refers to a non-aggregate nanoparticle, or one which is not composed of smaller nanoparticles. A primary carbon nanoparticle is formed directly from organic molecules, or organic and inorganic molecules. A primary carbon nanoparticle may comprise multiple different molecular building blocks (e.g. multiple organic and/or inorganic molecules), but cannot be reduced to smaller nanoparticles. The term “primary carbon nanoparticle” encompasses unmodified PCN; “surface-modified primary carbon nanoparticle” or “smPCN;” “surface-oxidized primary carbon nanoparticles” or “soPCN;” and “surface-functionalized primary carbon nanoparticles” or “sfPCN.”

As used herein, the term “surface-modified primary carbon nanoparticle” refers to a PCN in which all or a portion of its surface has been chemically modified, for example, by chemical reaction (e.g. oxidation), or the addition of functional groups or conjugation of one or more groups (e.g. chemical substituents, proteins, nucleic acids, lipids, carbohydrates, etc.) to the surface of the particle. “Surface-modified nanoparticles” encompass surface-oxidize nanoparticles (e.g. surface-oxidized primary carbon nanoparticles) as well as surface-functionalized nanoparticles (e.g. biomolecule conjugated primary carbon nanoparticles and/or biomolecule conjugated surface-oxidized primary carbon nanoparticles).

As used herein, the term “surface-oxidized primary carbon nanoparticle” or “soPCN” refers to a PCN in which all or a portion of its surface has been oxidized by chemical reaction (e.g. exposure to an oxidizing agent). One or more types of functional groups on the surface of a PCN are oxidized to yield a “soPCN.” A “soPCN” may be subjected to further chemical modification to yield a smPCN or subsequent conjugation to additional functional molecules (e.g. biomolecules) to yield sfPCN.

As used herein, the term “surface-functionalized primary carbon nanoparticle” or “sfPCN” refers to a PCN which has been modified by the addition of functional groups or conjugation to one or more groups (e.g. chemical substituents, proteins, nucleic acids, lipids, carbohydrates, etc.) to the surface of the particle. Modification or conjugation may occur at one or more types of functional groups on the surface of a PCN (e.g. oxidized sites, modified sites, etc.), soPCN, or smPCN.

As used herein, the term “monodisperse” refers to a sample containing particles wherein all of the particles contained therein are of substantially the same size (e.g. ±1%, ±2%, ±5%, ±10%, ±20%). Monodisperse particles have low polydispersity indices (e.g. 0.5 or less, 0.4 or less, 0.3 or less, 0.2 or less, 0.1 or less, 0.05 or less, etc.).

### DETAILED DESCRIPTION OF THE INVENTION

The present invention provides primary carbon nanoparticles (PCN), and methods of preparation and use thereof In particular, the present invention provides surface-modified PCN (smPCN). In some embodiments, the present invention provides monodisperse PCN and/or monodisperse smPCN. In particular, the present invention provides PCN and/or smPCN with diameters greater than 100 nm. In some embodiments, PCN are produced from carbon black (e.g. thermal black). In some embodiments, the present invention provides surface-oxidized carbon nanoparticles (e.g. soPCN). In some embodiments, the present invention provides surface-modified carbon nanoparticles (e.g. smPCN). In some embodiments, the present invention provides surface-functionalized carbon nanoparticles (e.g. sfPCN). In some embodiments, the present invention provides biomolecules (e.g. antibodies) conjugated to PCN and/or smPCN. In some embodiments, the present invention provides methods or surface modification or functionalization of primary carbon nanoparticles (e.g. conjugation of biomolecules (e.g. antibodies) to primary carbon nanoparticles).

In some embodiments, the present invention provides PCN, and methods for preparing PCN from reagents including one or more carbon precursors (e.g. amorphous carbon (e.g. carbon black (e.g. thermal black), soot, coal, etc.)). In some embodiments, the present invention provides compositions and methods for preparing PCN from carbon black. In some embodiments, carbon blacks include, but are not limited to any types of carbon blacks produced by the incomplete combustion or thermal decomposition of natural gas or petroleum oil. In some embodiments, primary carbon nanoparticles are prepared from any suitable carbon black, including, but not limited to: acetylene black, channel black, furnace black, lamp black and thermal black. Representative types of carbon blacks suitable for use in the present invention include, but are in no manner limited to, the Sp series of carbon blacks (e.g. from EVONIK) such as, but not limited to Sp-15, Sp-15/CIGHT, Sp4, Sp100, Sp250, Sp350; the N100, N200 and N300 series blacks such as, but not limited to, N110, N115, N121, N343, CD-2041, N326, N134, N220, N231, N234, N330, N339, N347, N375, CD-2013, CD-2014, CD-2015, CD-2016, CD-2005, OB-2015, HV-3396, RCB 2-17, N219, N242, N299, N351 and other ASTM grades; CABOT carbon blacks such as Mogul-E, VulcanXC72; etc. In some embodiments, primary carbon nanoparticles are prepared from thermal black (e.g. a low structured thermal black, AROSPERSE 15V, fine thermal black, medium thermal black, Sp15, etc.). In some embodiments, methods of the present invention produce monodisperse PCN from carbon black (e.g. thermal black). In some embodiments, the carbon black for use in the present invention is selected to achieve desired characteristics of the PCN produced (e.g. size, light absorption, etc.). In some embodiments, thermal black (e.g. fine thermal black, medium thermal black, etc.) is selected to produce PCN greater than 100 nm in diameter.

In some embodiments, PCN are at least 10 nm in diameter (e.g. 10 nm . . . 20 nm . . . 50 nm . . . 100 nm . . . 200 nm . . . 500 nm . . . 1000 nm, etc.). In some embodiments, PCN are at least 50 nm in diameter (e.g. 50 nm . . . 100 nm . . . 200 nm . . . 500 nm . . . 1000 nm, etc.). In some embodiments, PCN are at least 100 nm in diameter (e.g. 100-200 nm, 100-1000 nm, 100-500 nm, 120-200 nm, 150-250 nm, 200-400 nm, 300-700 nm, etc.). In some embodiments, nanoparticles of the present invention are monodisperse regardless of particle size. In some embodiments, nanoparticles of the present invention are non-aggregate (a.k.a. primary) regardless of particle size. In some embodiments, nanoparticles of the present invention are monodisperse and non-aggregate (a.k.a. primary). In some embodiments, the PCN have greater mass than similarly-sized aggregate carbon nanoparticles. In some embodiments, the PCN have increased light absorption when compared to similarly-sized aggregate carbon nanoparticles. In some embodiments, PCN absorb more light per particle than similarly-sized aggregate carbon nanoparticles, resulting in higher signal per binding event and consequently more sensitivity when deployed in a detection system.

In some embodiments, PCN produced by the methods of the present invention are monodispersed. In some embodiments, small polydispersity indices of PCN verify narrow particle size distribution. In some embodiments, particles of the present invention have polydispersity indices of less than 0.5 (e.g. 0.4 . . . 0.3 . . . 0.2 . . . 0.1, etc.). In some embodiments, particles of the present invention have polydispersity indices of about 0.05-0.5 (e.g. 0.05 . . . 0.1 . . . 0.2 . . . 0.25 . . . 0.3 . . . 0.4 . . . 0.5). In some embodiments, particles of the present invention have polydispersity indices of about 0.1, 0.2, 0.3, or 0.4. In some embodiments, the monodispersity of particles of the present invention allows for improved storage characteristics and/or processability in a manufacturing environment.

In some embodiments, PCN of present invention are surface-oxidized primary carbon nanoparticles (soPCN). In some embodiments, soPCN are prepared by exposure of carbon reagents (e.g. carbon black) and/or unoxidized PCN to an oxidizing agent (e.g. hydrogen peroxide). In some embodiments, soPCN are prepared by exposure of carbon reagents (e.g. carbon black) and/or unoxidized PCN to an oxidizing agent (e.g. hydrogen peroxide) and acid (e.g. strong acid (e.g. nitric acid)). In some embodiments, soPCN are prepared by exposure of carbon reagents (e.g. carbon black) and/or unoxidized PCN to an acidic and/or acidified oxidizing agent. In some embodiments, carbon reagents (e.g. thermal black) and/or unoxidized PCN are exposed to two or more oxidizing agents (e.g. hydrogen peroxide and nitric acid) to produce soPCN.

In some embodiments, suitable oxidizing agents for use in preparation of surface-oxidized primary carbon nanoparticles include: ammonium cerium(IV) nitrate, chlorite, chlorate, perchlorate, chromic acid, dichromic acids, chromium trioxide, pyridinium chlorochromate (PCC), chromate/dichromate compounds, hydrogen peroxide, hypochlorite, bleach, Iodine, nitric acid, nitrous oxide (N₂O), osmium tetroxide (OsO₄), ozone, permanganate salts, peroxide compounds, persulfuric acid, potassium nitrate (KNO₃), sulfoxides, sulfuric acid, Tollens' reagent, 2,2′-Dipyridyldisulfide, etc. In some embodiments, the present invention provides hydrogen peroxide and/or a hydrogen peroxide solution as an oxidizer. In some embodiments, a hydrogen peroxide solution is made from any commercially available source of hydrogen peroxide, and may be used in varying strengths (percents of hydrogen peroxide in aqueous solution). In some embodiments, from 1%, to 99% hydrogen peroxide solutions are used as oxidizers. In some embodiments of the present invention, aqueous hydrogen peroxide solutions comprise 1% . . . 2% . . . 5% . . . 10% . . . 20% . . . 30% . . . 50% . . . 75% . . . 90% . . . 99% hydrogen peroxide.

In some embodiments, the present invention provides an acid solution. In some embodiments, an acid solution comprises one or more acids (e.g. strong acids). In some embodiments, an acid solution is made from any commercially available source, and may be used in varying acidity and/or strengths (e.g. any pH, any molarity). In some embodiments, an acid solution has a pH of less than 7 (e.g. pH 6 . . . pH 5 . . . pH 4 . . . pH 3 . . . pH 2 . . . pH 1). In some embodiments, an acid solution has a concentration of 10 mM or greater (e.g. 10 mM . . . 20 mM . . . 50 mM . . . 100 mM . . . 200 mM . . . 500 mM . . . 1 M . . . 2 M . . . 3 M . . . 4 M . . . 5 M . . . 6 M . . . 7 M . . . 8M . . . 9 M . . . 10 M . . . 11 M . . . 12 M, etc.).

In some embodiments, suitable acids for use in preparation of surface-oxidized primary carbon nanoparticles include: hydroiodic acid, hydrobromic acid, perchloric acid, hydrochloric acid, sulfuric acid, p-toluenesulfonic acid, hydronium ion, nitric acid, chloric acid, bromic acid, perbromic acid, iodic acid, periodic acid, fluoroantimonic acid, magic acid, carborane superacid, fluorosulfuric acid, triflic acid, etc. In some embodiments, the present invention provides nitric acid and/or a nitric acid solution as an acid. In some embodiments, a nitric acid solution is made from any commercially available source, and may be used in varying acidity and/or strengths (e.g. any pH, any molarity). In some embodiments, a nitric acid solution has a pH of less than 7 (e.g. pH 6 . . . pH 5 . . . pH 4 . . . pH 3 . . . pH 2 . . . pH 1). In some embodiments, a nitric acid solution has a concentration of 10 mM or greater (e.g. 10 mM . . . 20 mM . . . 50 mM . . . 100 mM . . . 200 mM . . . 500 mM . . . 1 M . . . 2 M . . . 3 M . . . 4 M . . . 5 M . . . 6 M . . . 7 M . . . 8M . . . 9 M . . . 10 M . . . 11 M . . . 12 M, etc.).

In some embodiments, carbon reagents and/or unoxidized PCN are exposed to one or more oxidizers (e.g. hydrogen peroxide). In some embodiments, carbon reagents and/or unoxidized PCN are exposed to an oxidizer (e.g. hydrogen peroxide) and acid (e.g. strong acid (e.g. nitric acid)). In some embodiments, carbon reagents and/or unoxidized PCN are exposed to an oxidizer (e.g. hydrogen peroxide) and acid (e.g. strong acid (e.g. nitric acid)) under elevated temperature (e.g. 30° C. . . . 40° C. . . . 50° C. . . . 60° C. . . . 70° C. . . . 80 ° . . . 90° C., etc.). In some embodiments, carbon reagents and/or unoxidized PCN are exposed to hydrogen peroxide and nitric acid under elevated temperature (e.g. >50° C. (e.g. 85° C.)). In some embodiments, soPCN produced by the methods of the present invention exhibit higher carbon surface oxidation than carbon nanoparticles produced by other methods (e.g. standard or existing methods). In some embodiments, the increased surface oxidation of the PCN of the present invention provides better assay-performance characteristics (e.g. better initial dispersion in buffer etc.).

In some embodiments, surface oxidation of PCN provides reactive groups for further and/or subsequent functionalization (e.g. addition of functional groups, conjugation with biomolecules or polymers, etc.). In some embodiments, soPCN are reacted with one or more functional-group containing molecules to generate surface-functionalized primary carbon nanoparticles (sfPCN). In some embodiments, the surfaces of PCN are configured for functionalization with any functional group (e.g. reactive group), substituent, and/or molecule (e.g. polymer, biomolecule, label, etc.). In some embodiments, suitable functional groups for attachment to the surface of PCN and/or soPCN include, but are not limited to any functional groups comprising carbon, hydrogen, nitrogen, oxygen, phosphorous, sulfur, and/or halogen atoms, including but not limited to alkyl, alkenyl, alynyl, phenyl, halo-substituents (e.g. fluoro, chloro, bromo, iodo), haloformyl, hydroxyl, carbonyl, aldehyde, carbonate ester, carboxylate carboxyl, ether, ester, hydroperoxy, peroxy, carboxamide, amine (e.g. tertiary), imine, imide, azide, azo, cyanates, isocynates, nitrate, nitrile, nitrite, nitro, nitroso, pyridine, phosphine, phosphodiester, phosphonic acid, phosphate, sulfide, sulfone, sulfonic acid, sulfoxide, thiol, thiocyanate, disulfide, and any combination or derivative thereof. In some embodiments, the increased surface oxidation of the PCN of the present invention provides the presence of increased sites for surface functionalization. In some embodiments, the increased surface oxidation of the soPCN of the present invention provides the presence of more functional groups for subsequent biomolecule conjugation. In some embodiments, conjugation methods of the present invention provide more efficient addition of functional groups or biomolecules to the surface of PCN. In some embodiments, conjugation methods of the present invention provide addition of greater number of functional groups or biomolecules to the surface of PCN.

In some embodiments, the present invention provides biomolecules (e.g. antibodies), polymers, labels, etc. conjugated to PCN (e.g. soPCN, sfPCN, etc.). In some embodiments, the present invention provides methods of conjugating biomolecules (e.g. antibodies), polymers, labels, etc. to PCN. In some embodiments, the present invention provides biomolecules (e.g. antibodies) conjugated to PCN. In some embodiments, the present invention provides methods of conjugating biomolecules (e.g. antibodies) to PCN. In some embodiments, PCN (e.g. soPCN, sfPCN, etc.) are coated in biomolecules (e.g. antibodies). In some embodiments, the present invention provides methods of coating PCN in biomolecules (e.g. antibodies). In some embodiments, biomolecules are attached to surface functional groups on sfPCN. In some embodiments, a surface oxidation site on soPCN provides sites for biomolecule attachment. In some embodiments, biomolecules are attached to PCN by a linker. In some embodiments, suitable biomolecules include nucleic acids (e.g. DNA, RNA), proteins (antibodies, etc.), carbohydrates (e.g. polysaccharides), lipids, macromoloecular complexes, etc.

In some embodiments, the present invention provides linkers for attaching biomolecules to PCN. In some embodiments, a linker connects one biomolecule to one PCN. In some embodiments, a linker comprises any molecular element configured to attach one or more biomolecules (e.g. 1, 2, 3, 4, 5, 6, 7, 8, etc.) to one or more PCN (e.g. 1, 2, 3, 4, 5, 6, 7, 8, etc.). In some embodiments, more than one biomolecule is connected to a PCN by a linker In some embodiments, many biomolecules (e.g. >10, >100, >1000, etc.) are attached to a single PCN by linkers. In some embodiments, a linker is configured to attach one biomolecule to one PCN. In some embodiments, surface modifications (e.g. oxidation, functionalization, etc.) of PCN provide sites (e.g. many sites) for attachment of biomolecules through linkers. A wide variety of linkers may be used. In some embodiments, the linker is a single covalent bond. In some embodiments, the linker comprises a linear or branched, cyclic or heterocyclic, saturated or unsaturated, structure having 1-20 nonhydrogen atoms (e.g., C, N, P, O and S); and is composed of any combination of alkyl, ether, thioether, imine, carboxylic, amine, ester, carboxamide, sulfonamide, hydrazide bonds and aromatic or heteroaromatic bonds. In some embodiments, linkers are longer than 20 nonhydrogen atoms (e.g. 21 non-hydrogen atoms, 25 non-hydrogen atoms, 30 non-hydrogen atoms, 40 non-hydrogen atoms, 50 non-hydrogen atoms, 100 non-hydrogen atoms, etc.) In some embodiments, the linker comprises 1-50 non-hydrogen atoms (in addition to hydrogen atoms) selected from the group of C, N, P, O and S (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, or 50 non-hydrogen atoms). In some embodiments, the linker comprises an alkyl chain. In some embodiments, the linker comprises a polymer (e.g. nucleic acid, polypeptide, lipid, polysaccharide, synthetic polymer, PEG, etc.). In some embodiments, a linker comprises one or more additional substituents. In some embodiments, the linker is attached covalently at one end to a PCN and at a second end to a biomolecule. In some embodiments, a linker is enzyme cleavable, such that exposure to specified enzyme cleaves the linker and separates the biomolecule from the PCN. In some embodiments, a linker is physically or chemically cleavable, such that exposure to specified conditions (e.g. UV light, heat, pH, etc.) cleaves the linker and separates the biomolecule from the PCN. One of ordinary skill in the art will further appreciate that the above linkers are not intended to be limiting. Any linker suitable for connecting the two moieties can be used.

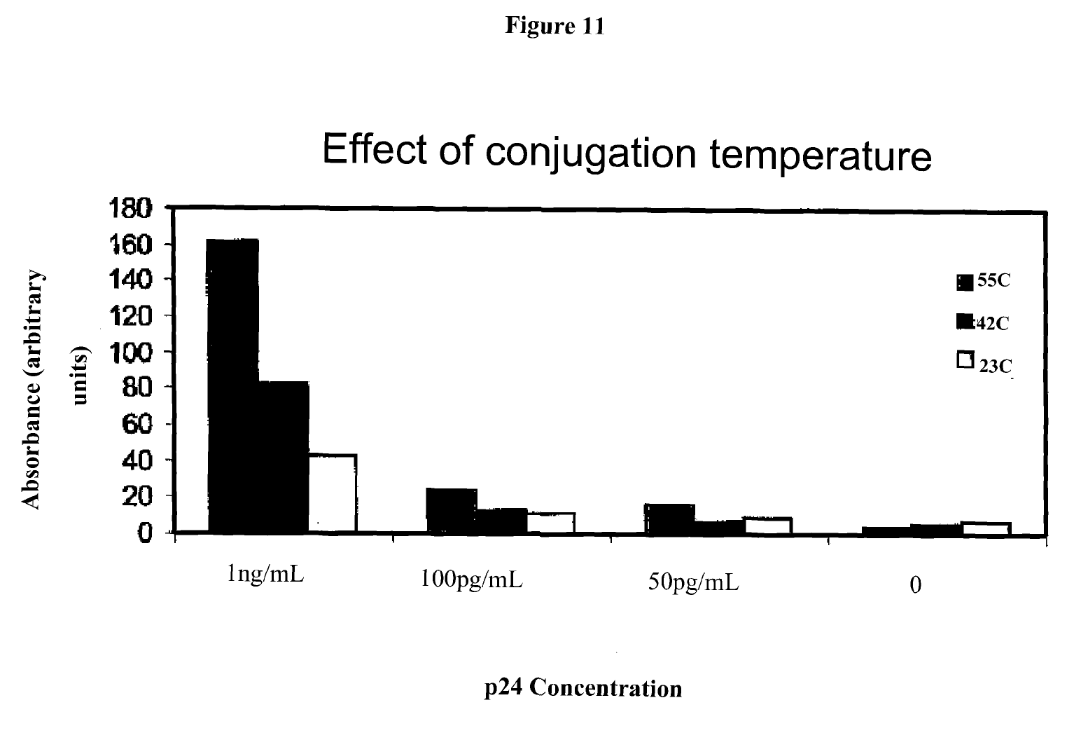

In some embodiments, PCN of the present invention are conjugated to and/or coated with biomolecules and/or linker groups at high temperature (e.g. 50° C. . . . 60° C. . . . 90° C., etc.). In some embodiments PCN and one or more biomolecules are conjugated at a temperature above physiological temperature. In some embodiments PCN and one or more biomolecules are conjugated above the physiological temperature of the species or the hybridoma from which the biomolecule (e.g. antibody) was derived. In some embodiments, increased temperature increases the rate of biomolecule adhesion to the surface. In some embodiments, high temperature coating enhances orientation of the biomolecules for more efficient molecular recognition events. In some embodiments, high temperature coating is also known as “hot start conjugation.” In some embodiments, high temperature coating allows for improved distribution of particles in solution. In some embodiments, high temperature coating allows for proper dispersion (e.g. not polydisperse) through increased molecular motion in solution. In some embodiments, high temperature coating increases the rate of adhesion of molecules (e.g. biomolecules) to the particle (e.g. PCN) surface. In some embodiments, high temperature coating reduces background signal in assays in which the smPCN (e.g. sfPCN) are involved (SEE FIG. 11). In some embodiments, high temperature coating decreases the rate and/or occurrence of particle self-aggregation. In some embodiments, high temperature coating allows for partial and/or total denaturation of biomolecules, thereby exposing functional and/or reactive groups (e.g. residues embedded in the internal structure of a protein (e.g. hydrophobic residues on an antibody)) for adhesion to PCN. In some embodiments, high temperature coating increases the rate of adhesion, thereby decreasing total incubation time. In some embodiments, high temperature coating results in more complete coating of PCN. In some embodiments, high temperature coating increases the degree of surface conjugation (e.g. increases ratio of bound to unbound surface accessible functional groups (e.g. sites of oxidation)). In some embodiments, high temperature coating results in increased shelf life and improved storage characteristics.

In some embodiments, smPCN, soPCN, sfPCN and/or biomolecule-conjugated PCN are monodisperse. In some embodiments, smPCN, soPCN, sfPCN and/or biomolecule-conjugated PCN exhibit polydispersity indices of less than about 0.5 (e.g. about 0.4, about 0.3 about 0.2 about 0.1, less than 0.1). In some embodiments, smPCN, soPCN, sfPCN and/or biomolecule-conjugated PCN have diameters, including functional groups and/or biomolecules of greater than about 100 nm (e.g. 150 nm . . . 200 nm . . . 300 nm . . . 400 nm . . . 500 nm . . . 600 nm . . . 700 nm . . . 800 nm . . . 900 nm . . . 1000 nm, etc.). In some embodiments, surface functional groups and/or surface-conjugated biomolecules increase the diameter of a PCN. In some embodiments, PCN are oxifized, surface-functionalized, surface-modified, and/or conjugated to biomolecules without resulting in aggregartion.

In some embodiments, PCN, smPCN, soPCN, sfPCN, and/or biomolecule-conjugated PCN find use in a variety of research, environmental, biomedical, clinical, diagnostic, veterinary, and materials applications. In some embodiments, primary carbon nanoparticles of the present invention find use in lateral flow assays and/or molecular detection systems, such as those described in Internat. Pub. No. WO 2004/045381, U.S. Pat. App. No. 2008/0138842, U.S. Pat. No. 7,384,796, U.S. Pat. No. 7,465,587, U.S. Pat. No. 6,737,278, and U.S. Pat. No. 6,120,594, herein incorporated by reference in their entireties. In some embodiments, a lateral flow assay employs a labeled binding reagent comprising a colored particle and a binding portion capable of binding the analyte. A PCN conjugated to a biomolecule (e.g., antibody) as described herein may be used as the labeled binding reagent comprising a colored particle and a binding portion capable of binding the analyte in a lateral flow assay. Analyte present in a liquid sample can participate in a sandwich or a competition reaction within a detection zone of a lateral flow device, with the PCN, which can also be incorporated in a test strip or applied thereto. The smPCN (e.g., surface-modified with an antibody) may be disposed in the macroporous body in a dry state and may further be mobilizable by a liquid sample that passes along a flow path. In one aspect, the PCN is configured to competitively form a complex with analyte present in the liquid sample or with the analyte or the analogue thereof immobilized in the detection zone. The smPCN (e.g., surface-modified with an antibody), or a plurality of such PCN, may be employed in a lateral flow assay in such a manner to produce an instant analytical result that is readily visible to the eye (or an optical detector) if it becomes bound in the detection zone. In some embodiments, PCN of the present invention are not limited to any applications described herein. A skilled artisan will appreciate numerous additional applications for the compositions and methods described herein. For example, PCN, soPCN, smPCN, and sfPCN (e.g. antibody functionalized PCN) of the present invention find use in therapeutic applications, e.g., as a therapeutic or therapeutic delivery compositions. In some embodiments, compositions of the present invention find use in localized heating (e.g. tissue heating) through light, photochemical, or binding activation of the PCN. In some embodiments, the light absorptive properties of compositions of the present invention provide localized heating (e.g. tissue heating) in an area in which PCN are deployed (e.g. in vitro or in vivo).

### Experimental

The following section provides exemplary embodiments of the present invention, and should not be considered to be limiting of its scope with regard to alternative embodiments that are not explicitly described herein.

### EXAMPLE 1

### Carbon Oxidation

5 g of carbon (AROSPERSE 15) was transferred into a 40 mL glass vial. 7 mL of water was added and the temperature was maintained at 85° C. in a water bath. 3 mL of 30% hydrogen peroxide was added, followed by 200 μL of 16M nitric acid. The reaction was kept at 85° C. for 5 hours after which 350 mg of sodium bicarbonate was added in to the solution to neutralize the remaining acid. The sample was then transferred into 50 mL polypropylene tubes and washed 3 times by centrifugation at 5000 rpm and resuspended in water. The pellet was transferred into a glass vial and dried for three days in a 60° C. oven. 4.9 grams of black chalky material was recovered.

### EXAMPLE 2

### Primary Carbon Nanoparticle Characterization

Experiments were performed during development of embodiments of the present invention to analyze the composition of primary carbon nanoparticles by X-ray photoelectron spectroscopy (XPS). Composition analysis revealed that the commercially available SP15 has a carbon:oxygen ration that falls in between SP-4 and SP-100 (two previously used carbon blacks with smaller particle sizes of 25 nm and 56 nm, respectively). However, upon further oxidation via hydrogen peroxide (using the oxidation method described in U.S. Pat. No. 6,120,594), SP15-O CIGHT exhibits the highest oxygen content (e.g. carbon:oxygen ratio of 11.5:1 or greater (e.g. 11.9:1)). Detectable amounts of Silicon were also identified in the both Sp-15 samples using the XPS method before and after oxidation (C:O:Si=11.9:1:0.16). In some embodiments, the source of the silicon is the furnace brick used in the production of carbon black and or thermal black by certain production practices.

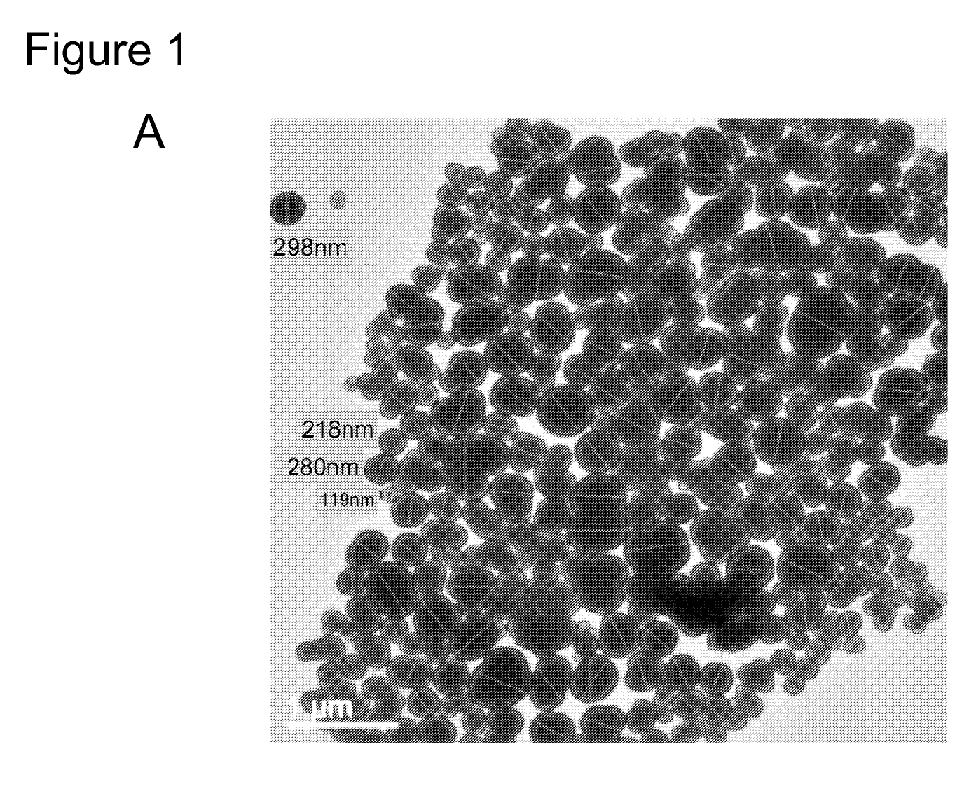

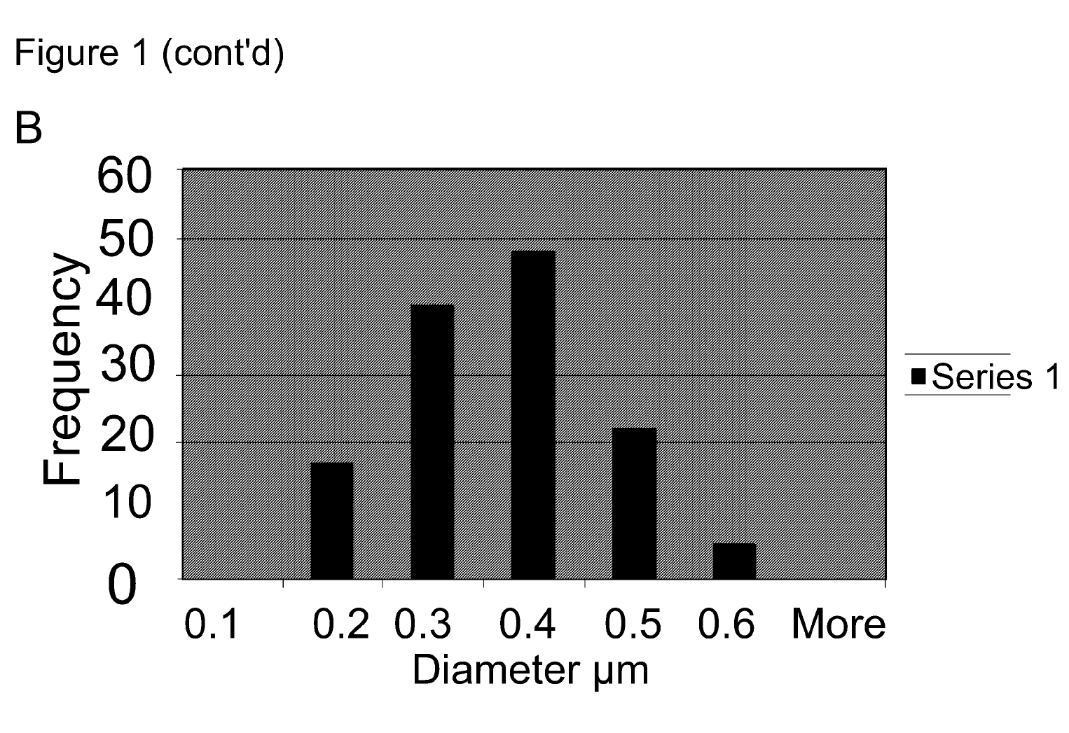

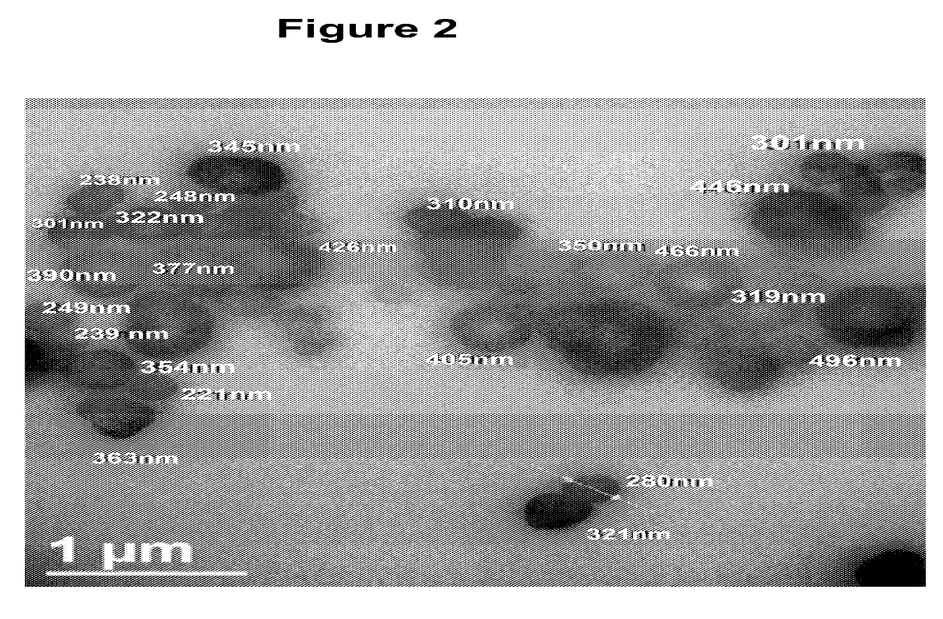

Experiments were performed during development of embodiments of the present invention to determine the size distribution of the oxidized and unconjugated Sp-15, as well as the antibody conjugated particles, using transmission electron microscopy (SEE FIGS. 1-2). The average particle size was determined to be 315 nm with 87% of particles being larger than 200 nm with a range of 119-570 nm (SEE FIG. 1B). The primary particle size in this example is greater than 100 nm for the smallest particle present. The TEM measurements were carried out on Hitachi H8100 instrument at the NWU Nuance facility.

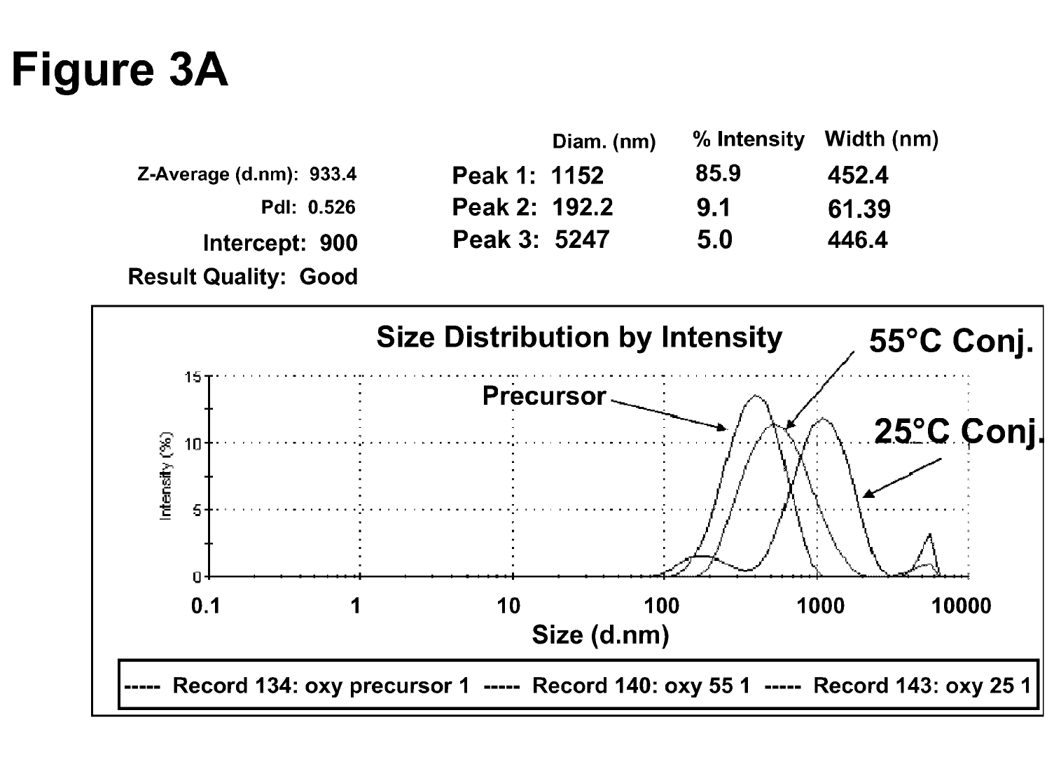

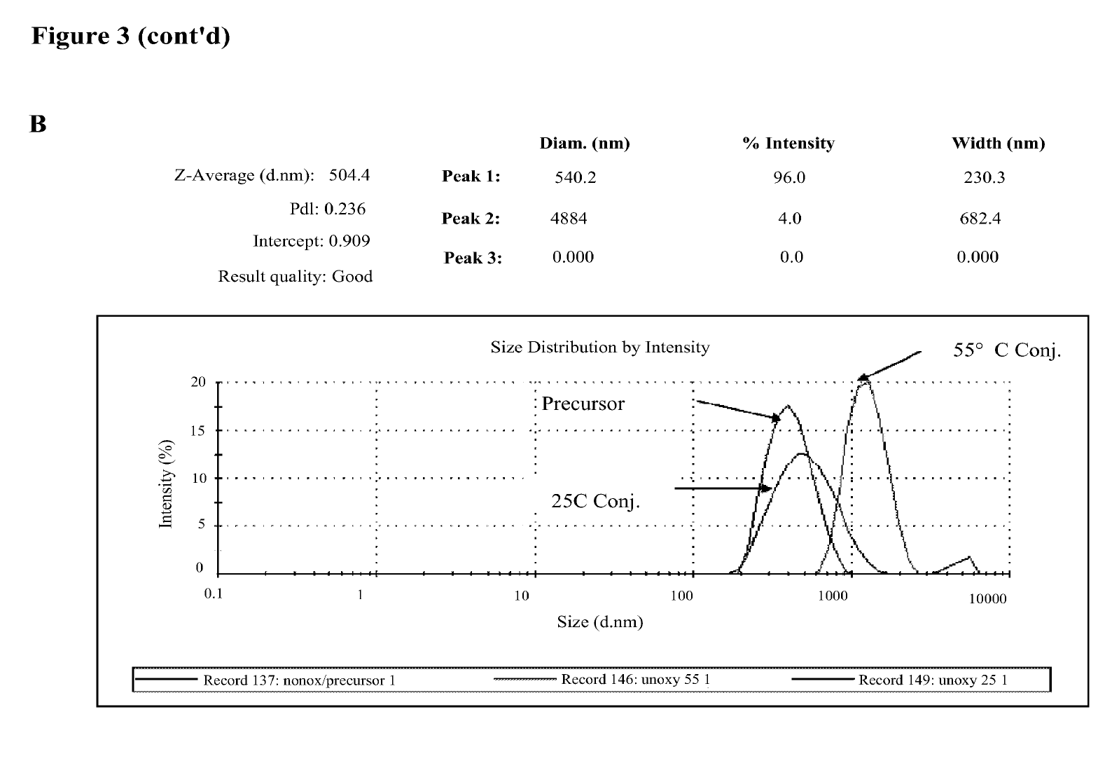

Experiments were conducted during development of embodiments of the present invention using dynamic light scattering to determine the size of carbon conjugates produced using various conjugation methodologies (SEE FIG. 3). Particles were diluted 4 fold in water and analyzed using Zeta Sizer Nano-ZS (Malvern). Particle size was estimated using intensity analysis using three separate measurements. Oxidized carbon nanoparticles produced from precursor with a 356 nm mean diameter and 0.15 polydispersity exhibited 936 nm mean diameter and 0.5 polydispersity index after antibody conjugation at 25° C.; and 525 nm mean diameter and 0.2 polydispersity index after antibody conjugation at 55° C. Non-oxidized carbon nanoparticles produced from precursor with a 386 nm mean diameter and 0.1 polydispersity exhibited 503 nm mean diameter and 0.2 polydispersity index after antibody conjugation at 25° C.; and 1347 nm mean diameter and 0.2 polydispersity index after antibody conjugation at 55° C.

Functional testing in spiked human plasma samples was conducted during development of embodiments of the present invention to evaluate Sp15 conjugates for use in a p24 dipstick assay (See e.g., U.S. Pat. No. 7,713,746). Carbon conjugates can be used as an effective detector molecule in lateral flow assays due to their high absorbance and good contrast with the white nitrocellulose background. However, conjugation methodologies can produce conjugates with varying properties such as monodispersity and antibody coating density. To optimize conjugation methodologies of the present invention, carbon-antibody conjugates were produced using oxidized (using oxidation method described in U.S. Pat. No. 6,120,594) and non-oxidized carbon nanoparticles (SP-15) at 25° C. or 55° C. and then evaluated in a p24 dipstick assay.

Standards were prepared by serially diluting recombinant p24 antigen into seronegative adult plasma. 25 uL aliquots of each standard were diluted with 75 uL heat shock buffer and heated for 4 minutes at 88° C. After allowing the samples to cool to room temperature, 3 uL biotinylated mAb115B antibody and 3-9 uL carbon conjugate were added. The volume of carbon conjugate was varied to adjust for concentration differences as measured using Cary 500 spectrophotometer (1=500 nm).

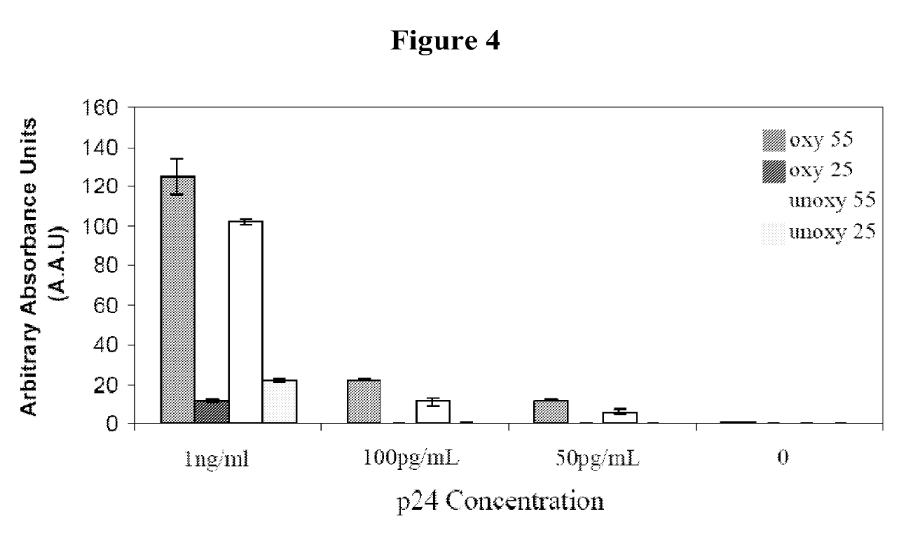

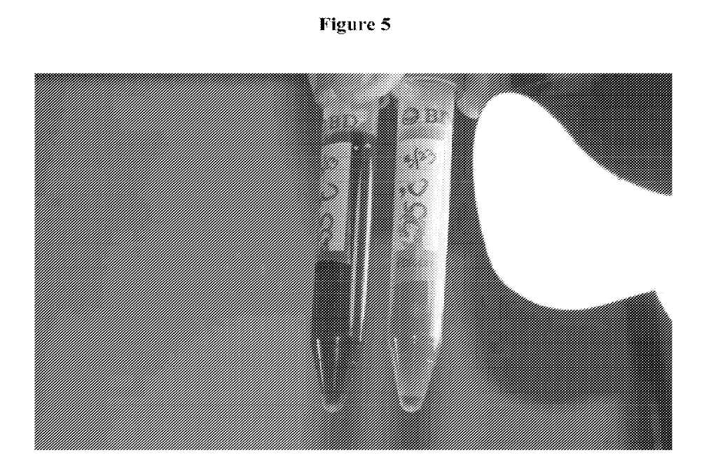

It was observed that the conjugates prepared at 55° C. performed superior to those prepared at 25° C. (SEE FIG. 4). The former produced reaction strips with strong control lines, while those at 25° C. gave weaker lines. In addition, unoxidized particles prepared at 55° C. were only usable on the first day after which precipitated upon standing. The actual examples of the images of performed experiments are provided in the following two pages.

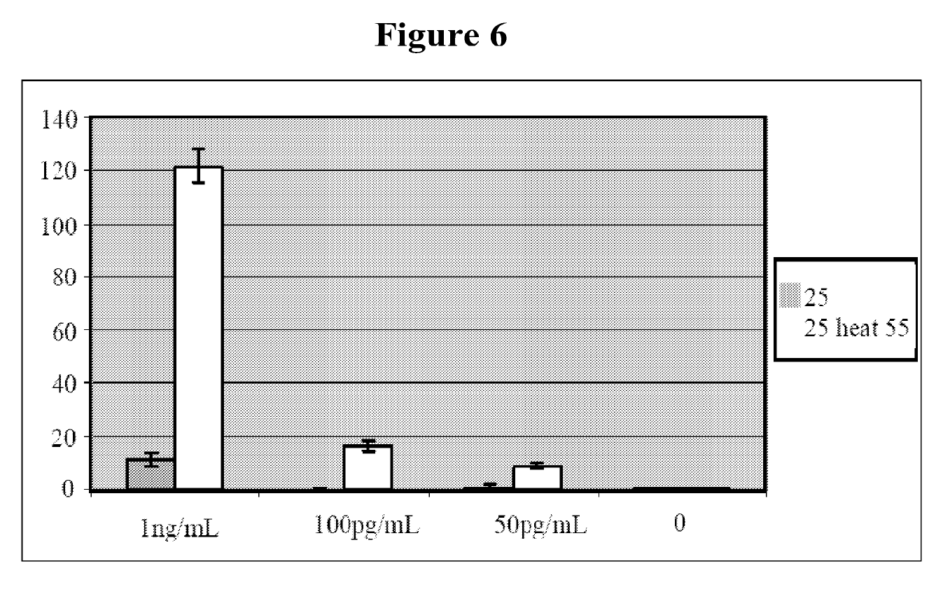

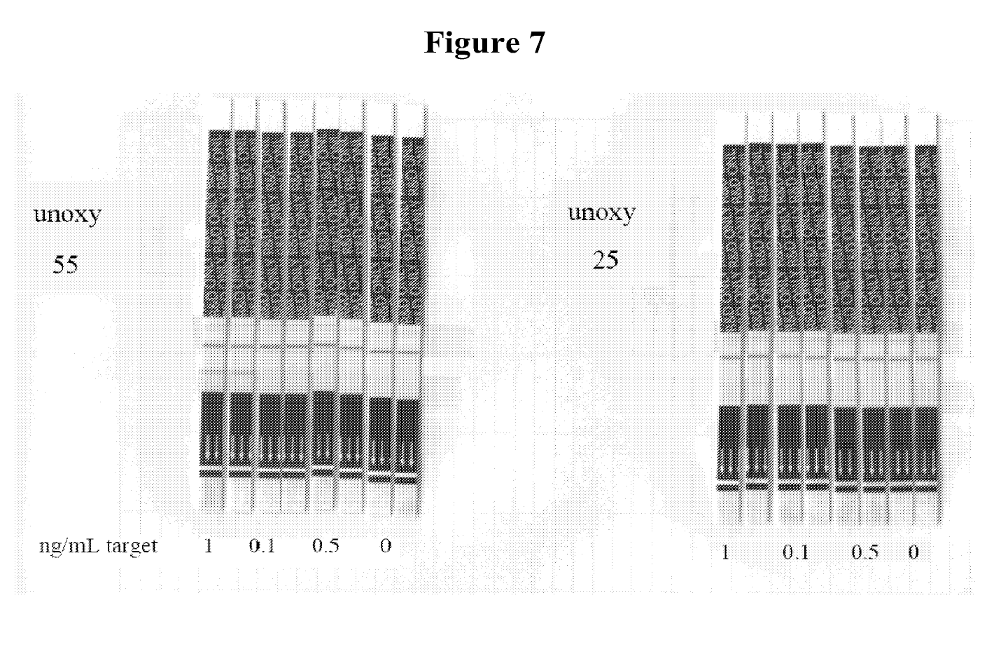

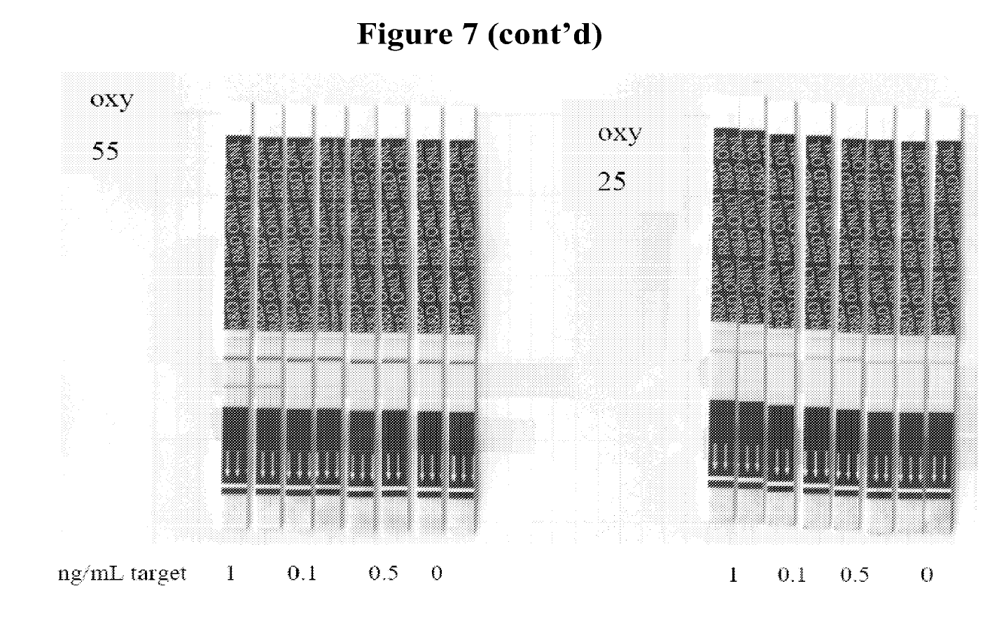

It was demonstrated that non functional particles (those prepared at 25° C. can be rendered functional by heating them in an oven at 55° C. for two hours (SEE FIG. 6). Upon doing so they retain all the characteristics of particles prepared at 55° C. Although the present invention is not limited to any particular mechanism of action and an understanding of the mechanism of action is not necessary to practice the present invention, it is contemplated that reorientation of the Ab to an extent that exposes the adhesive functional group upon partial denaturation of the Ab is responsible for this effect. The latter effect allows for the proper conjugation of the Ab to the particles when the conjugation and passivation are both is carried out at 55° C.

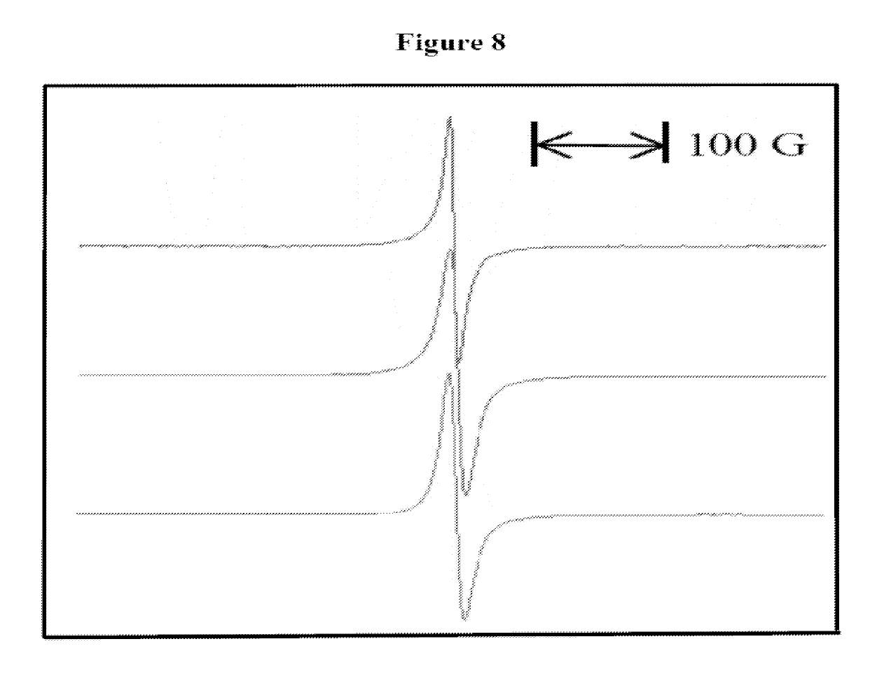

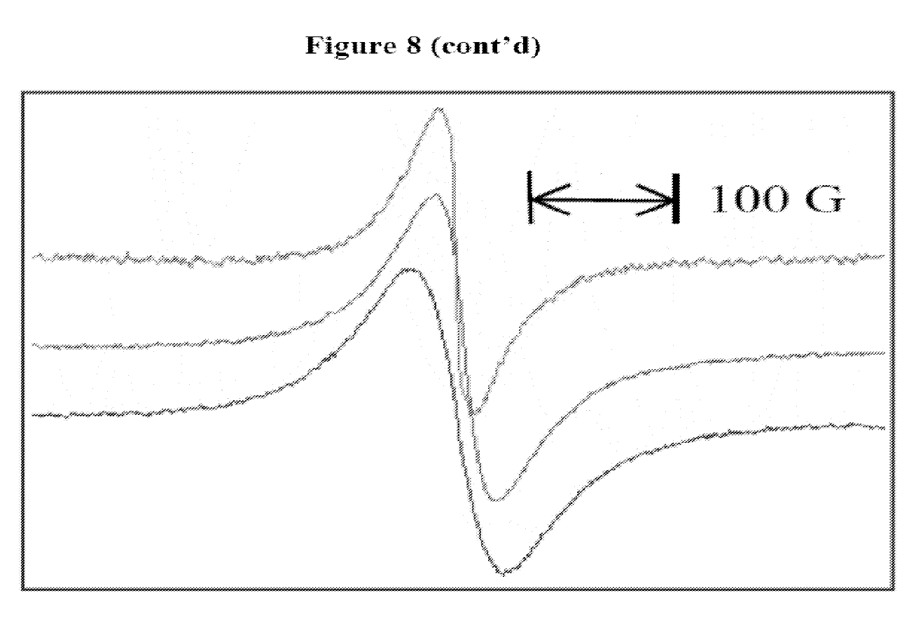

Various carbon black samples were characterized by Electron Paramagnetic Resonance (EPR) spectroscopy at two different microwave frequencies: 9.3 GHz (X band) and 34.0 GHz (Q band). X-band spectra were obtained on solid samples both at room temperature (295 K) and liquid nitrogen temperature (77 K). All Q-band spectra were obtained at room temperature on solid samples. The X-band RT spectra of the various carbon blacks shows that the samples can be broken into two rough categories of behaviors: narrow, linewidths (lw)<10 G p-p are seen for SP-15, Mogul E, and SP-350 (SEE FIG. 8A); broader lines with line width up to 100 G p-p are seen for SP-4, Regal, and SP-100 (SEE FIG. 8B). All spectra are centered on the free-electron g value of 2.00. Oxidation of SP-15 with H2O2 showed no change in the EPR lineshape of this sample. Little change in lineshape was observed for both classes when spectra were obtained at 77 K. EPR spectra of the narrow-line class of spectra were obtained at 34.0 GHz. The linewidths are similar to those seen at 9.3 GHz which strongly suggests that in these narrow line samples, the predominant broadening mechanism is homogeneous (life time) broadening rather than inhomogeneous (electron environment) broadening.

### EXAMPLE 3

### Characterization of Primary Carbon Nanoparticles

Experiments were conducted during development of embodiments of the present invention to characterize primary carbon nanoparticles produced by methods of the present invention (e.g. peroxide/acid/base method). XPS measurements, performed on an Omicron ESCAprobe with Al Kalpha X-ray, demonstrated the the elements C, O, Si, and Na were present in the nanoparticles. The ratio of the elements was C:O:Si:Na=11.5:1:0.008:0.018. Although the present invention is not limited to any particular mechanism of action and an understanding of the mechanism of action is not necessary to practice the present invention, it is contemplated that traces of sodium are derived from the base quencher, and silicon comes from the furnace brick that is used in the thermal black manufacturing process.

### EXAMPLE 4

### Preparation of Oxidized Carbon Particles

In some embodiments, carbon nanoparticles for subsequent functionalization and/or surface conjugation are oxidized and/or modified using methodologies described in U.S. Pat. No. 6,120,594.

36 grams of carbon black from Evonik Product Arosperse 15 beads was transferred to a crystallizing dish equipped with a large watch glass. 100 mL of 30% hydrogen peroxide was added at room temperature to create a suspension of carbon particles. This mixture was then placed on a hot plate and heated to boiling around 100° C. for one hour. The watch glass was then removed and the hydrogen peroxide was allowed to evaporate to dryness. The vessel was weighed to a constant weight for a period of 30 minutes after complete removal of all liquids. The contents of the dish were transferred to a balance and 35.2 grams of black/grey material was recovered. This material was then used in preparation of carbon conjugates (e.g. for diagnostics applications).

### EXAMPLE 5

### Conjugation of Antibodies to Carbon Nanoparticles

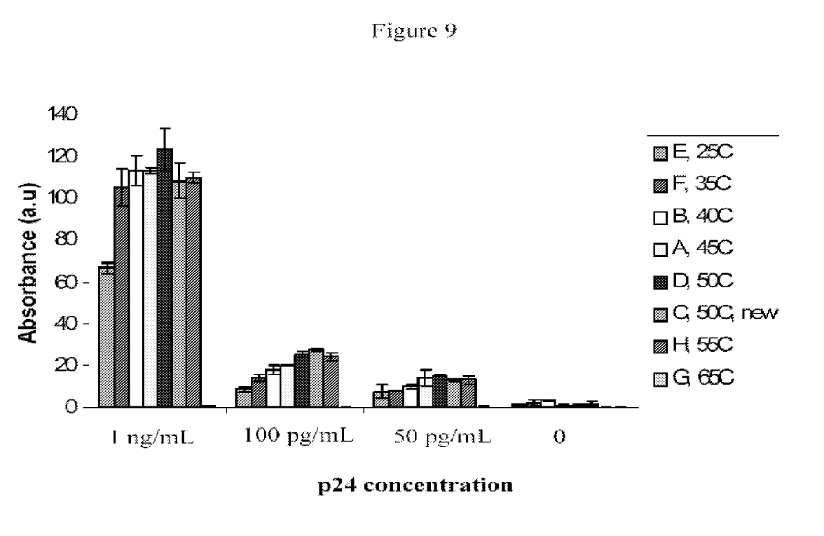

Experiments were conducted during development of embodiments of the present invention to conjugate biomolecules to carbon nanoparticles. A temperature study was performed on 280 nm carbon nanoparticles conjugates having monoclonal antibodies to demonstrate the effectiveness of a hot start process. The same number of carbon nanoparticles was incubated in conjugation buffer with the same concentration of antibody at different temperatures. After an hour, a solution of 10% BSA was introduced at the desired temperature to stop the reaction and allowed to incubate for an additional hour. The resulting particles were harvested by centrifugation and subjected to a lateral flow assay to determine activity and assess the background value in the assay using HIV antigen p24 as a target in prescreened human plasma. FIG. 9 shows the combined results from the lateral flow assays performed on the particles prepared in the aforementioned manner at different temperatures.

### EXAMPLE 6

### Prostate Specific Antigen (PSA) Detection

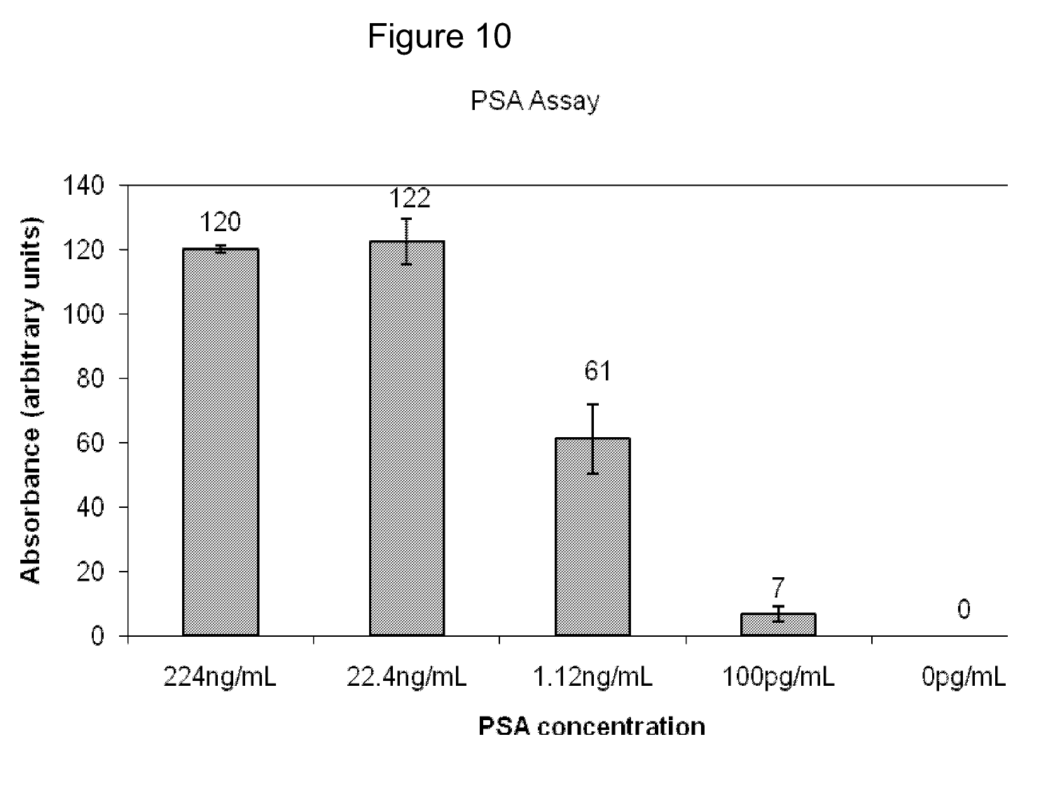

SP15 PCN were coated at 55° C. using one monoclonal and used in a sandwich assay with another monoclonal antibody acting as capture on nitrocellulose. The results are shown in FIG. 10.

### EXAMPLE 7

### Activity of Neutravidin Conjugates

PCN were coated with neutravidin at 55° C. A biotinylated antibody was added nitrocellulose having neutravidin capture immobilized thereon. The PCN-neutravidin conjugate was then added to the nitrocellulose to be captured at the immobilized biotinylated antibody. The control experiment lacked the biotinylated Ab. The neutravidin PCN were bound by the biotinylated antibody. The neutravidin PCN demonstrate the applicability of sbPCN to function with a variety of surface modifiers. The neutravidin PCN, and other related surface coated PCN, have utility in nucleic acids detection on lateral flow when the analyte is biotinylated.

### EXAMPLE 8

### Characterization of PCN Produced by a Peroxide/Acid/Base Method

Experiments were conducted during development of embodiments, of the present invention to characterize surface-oxidized PCN produced by a method in which thermal black is exposed to peroxide and nitric acid, and allowed to react therein, followed addition of a base quenching reagent. XPS data demonstrated a carbon:oxygen ratio of 11.5:1 with traces of sodium (e.g. from the base) and silicon (e.g. from the furnace brick used in production of the thermal black).

1.-47. (canceled)

48. A composition comprising monodisperse primary carbon nanopartices with diameters of about 100 nm or greater.

49. The composition of claim 48, wherein said primary carbon nanopartices have a polydispersity index of about 0.2.

50. The composition of claim 48, wherein said primary carbon nanopartices are between 200 nm and 600 nm in diameter.

51. The composition of claim 48, wherein said primary carbon nanopartices comprise an oxidized surface.

52. The composition of claim 48, wherein said primary carbon nanopartices are surface functionalized.

53. The composition of claim 52, wherein said primary carbon nanopartices are surface functionalized with one or more biomolecules.

54. A method of producing monodisperse primary carbon nanopartices comprising:
a) providing:
i) a carbon reagent;
ii) an oxidizing agent;
iii) a strong acid; and
iv) a basic quenching agent;
b) mixing said carbon, said oxidizing agent, and said strong acid;
c) heating the mixture; and
d) quenching the reaction with said basic quenching agent.

55. The method of claim 54, wherein said oxidizing agent comprises hydrogen peroxide.

56. The method of claim 54, wherein said strong acid comprises nitric acid.

57. The method of claim 54, wherein said basic quenching agent comprises sodium bicarbonate.

58. The method of claim 54, wherein said heating comprises heating at about 85° C.

59. Monodisperse primary carbon nanopartices produced by the method of claim 54.

60. The monodisperse primary carbon nanopartices of claim 59, wherein said primary carbon nanopartices have a polydispersity index of about 0.1-0.3.

61. The monodisperse primary carbon nanopartices of claim 59, wherein said primary carbon nanopartices are 200-600 nm in diameter.

62. A method of surface-functionalizing a monodisperse primary carbon nanoparticle comprising reacting surface-oxidized monodisperse primary carbon nanoparticles with one or more functional molecules at elevated temperature.

63. The method of claim 62, wherein said surface-oxidized monodisperse primary carbon nanoparticles comprise surface-oxidized thermal black.

64. The method of claim 62, wherein said elevated temperature is about 55-85° C.

65. The method of claim 62, wherein said functional molecule comprises a protein, peptide, polypeptide, or antibody.

66. The method of claim 62, wherein said functional molecule is directly conjugated to said surface-oxidized monodisperse primary carbon nanoparticles.

67. The method of claim 62, wherein said functional molecule is conjugated to said surface-oxidized monodisperse primary carbon nanoparticles by a linker.
